(19) United States
(12) Patent Application Publication
(10) Pub. No.: US 2022/0392583 A1
(43) Pub. Date: Dec. 8, 2022

(54) SYSTEM FOR TRAINING AN ENSEMBLE NEURAL NETWORK DEVICE TO ASSESS PREDICTIVE UNCERTAINTY

(71) Applicant: Firmenich SA, Satigny (CH)

(72) Inventors: Guillaume GODIN, Satigny (CH); Ruud VAN DEURSEN, Satigny (CH); Florian RAVASI, Morges (CH); Julien HERZEN, Morges (CH)

(21) Appl. No.: 17/835,845

(22) Filed: Jun. 8, 2022

(30) Foreign Application Priority Data
Jun. 8, 2021 (EP) 21178250.3

(51) Int. Cl. G16C 20/50; G06N 3/04; G16C 20/70

(52) U.S. Cl. CPC G16C 20/50; G06N 3/0472; G06N 3/0454; G16C 20/70; G06N 3/08

(57) ABSTRACT

The system (200) for training an ensemble neural network device configured to execute the steps of:
providing (205) a set of exemplar data, comprising at least one set of inputs (220) and at least one set of outputs (225) associated to the set of inputs, to a neural network device comprising an ensemble (230) of neural network devices, configured to provide independent predictions based upon the exemplar data,
operating (210) the neural network device based upon the set of exemplar data,
obtaining (215) the trained neural network device configured to provide an output,
the neural network device further comprising at least two independent activation functions, whereof at least two of the independent activation functions are representative of the statistical distribution of the plurality of independent predictions, the neural network device being configured to provide at least one output (235, 236) for at least two said independent activation functions and
the step of operating further comprising a step of operating each neural network device of the ensemble to provide an ensemble of outputs, the neural network device being trained to minimize the value representative of at least two said independent activation functions.

### TECHNICAL FIELD OF THE INVENTION

The present invention aims at a system for training a neural network device, a computer-implemented method to train a neural network device, a corresponding computer implemented neural network device, a computer program product, to execute the steps of the method object of the present invention, a computer-readable medium storing instructions to execute the steps of the method object of the present invention, a computer implemented method to predict a physical, chemical, medicinal, sensorial or pharmaceutical property of flavor, fragrance and drug ingredients and a computer implemented method to predict a category of representation in an image.

It applies, in particular, to the industry of flavors, fragrances and drugs or image processing applicable to the biological, chemical, medical, and pharmaceutical industries.

### BACKGROUND OF THE INVENTION

In scientific experiments, measurements 105 stored in databases, such as shown in FIG. 1, vary due to the environment of said experiment. One typically needs to use an ideal environment such as the international space station to get stable conditions. Even in near perfect environmental conditions, instruments and sample preparation by technicians may slightly vary from one to another. Merging experimental data from different sources can be difficult because of changing experimental variations depending on measurement conditions used. Statistical methods to homogenize experimental data have been developed to reduce those variations with moderate to good success. In the field of machine learning, such variations may exist between training and test sets as well as between the known and future data.

Another well-known issue of machine learning models is the number of hyperparameters in the model, which may significantly influence a model's ability to overfit the training data 110, such as shown in FIG. 1. The amount of data available may be insufficient to train the number of hyperparameters in a neural network. Therefore, it is best to consider the minimum need to extract a meaningful digital data representation for the data. The latter can be achieved by privileging layers with a lower number of parameters. An example of such an attempt is replacing dense layers by compact convolution layers. Convolution layers are particularly dedicated to extract from data localized features like images. However, even by using compact and efficient convolutional layers, networks with billions of parameters may still be generated. Trending mega-models show that such models can only be trained with very large datasets, which are, for instance, available for images, literature, and music. On the contrary, scientific fields like chemistry and biology frequently have datasets with some hundreds to thousands of data points. In these domains one should thus aim at a reduction on the number of parameters to match the size of the data.

One way to compensate for the large size of the networks is by data augmentation. Indeed, increasing performances linked to an increasing augmentation rate indicates that more data is needed for the selected size of the network, suggesting that the size of the network can be possibly reduced. Simultaneously, augmentation can be used to identify if a network is critically parametrized, i.e., the point where augmentation has no or little effect on the model's performance. Not all models are open to data augmentation. Graph neural networks (GNN), for instance, are invariant to representation shuffle. GNNs are thus incompatible with existing data augmentation methods used for natural language processing or images.

A third issue is that the training procedure 115 of a model defines an important aspect in modelling. Frequently, there are too many variables that may influence a model's decision. This may explain why hyper-parametrization optimization strategies may be required to improve models for performance or efficiency. Apart from the selected model, the question of the data split between train and test sets also plays a significant role. Several methods can be used from fully leave-one out, random-split to K-fold cross-validation to simulate and estimate the model quality on unseen data. In the end, a model's prediction is just an educated guess depending on the used training conditions, model size, optimization parameters, and data split. Upon completion, it is impossible to know if the best model was indeed trained. We assume, however, that the computed model is the best model for the used testing points. This is a general limitation of a data modeling approach as we should not necessarily expect that the performance results will be the same for all of parts of future unseen data. It should be noted that future performances may also vary considerably, depending on the evaluated sample size for the unseen data as well as a possible sample bias introduced in the unseen data. One way to partially solve these shortcomings is by predicting an accurate theoretical endpoint as standardized metric to evaluate a model. An example of such endpoint is the molecular weight of a molecule in chemistry.

In the last decades, deep neural networks (DNN) have become an essential part of modern live. Besides being a key technology in many scientific fields, DNNs are now gradually delivered as solutions for real-world applications. Neural networks are already successively applied in the field of medical imagery, image recognition and autonomously driving vehicles. Following the rapid growth of number of applications, it is crucial to reflect on some of the key shortcomings of neural networks. These shortcomings primarily include:
the lack of expressiveness and transparency on a model's decision logic,
the inability to distinguish between in-domain and out-of-domain predictions, including sensitivity to domain shifts,
the sensitivity to adversarial examples leading to neural networks that may be vulnerable to adversarial attacks and/or
the inability to provide a reliable estimate of the model's uncertainty.

Depending on scientific domain, requests to provide an explanation on the prediction of a neural network is a frequently mentioned topic. In fact, deep neural networks are typically perceived as black box technology. However, it should not be considered an essential shortcoming for its acceptance in applications. It should further be noted, that multiple similarly performing solutions can be generated and the machine's deduced logic may vary. The latter particularly applies between identical “machines” or models with different random initialisations. The explanation may thus vary between “machines”, and one consequently cannot judge whether the explanation is the best possible explanation. In other words, the deduced logic is a property of the trained system and it is one possible explanation.

In machine learning and artificial intelligence, it is well-known that a model's predictive performance may vary significantly between in-domain and out-of-domain points. In the case of a linear regression on a set of (x,y)-points, the modelled mathematical function y=f(x) should only be applied to all points that fall between the known points used to define the function. Out-of-domain predictions are extrapolations to the known data and the behaviour of data outside is unknown. It has been suggested (for example in the publication Applicability Domain: Towards a More Formal Framework to Express the Applicability of a Model and the Confidence in Individual Predictions, by Hanser & al., Advances in Computational Toxicology, pp 215-232) that these predictions should be systematically flagged or discarded. A model's applicability domain is further simultaneously defined by the model's architecture and by the data used to train the model. Consequently, the combined system of architecture and data defines a closed system and is an example of an undefinable problem. Using this one system, one can therefore not simultaneously tell the contributing factor of an out-of-domain prediction, i.e., whether it originates from the architecture or from the data. Consequently, this does not mean that a prediction will be out-of-domain for all systems. To have an out-of-domain assessment, one typically applies a mechanism like distance to the training set, statistical hoteling on the latent space, or the use of evidential layers. It should be noted that for those metrics the in-domain and out-of-domain calls become a result from the model and the selected domain method. In a stricter sense, one can even consider that the fact that a prediction is out-of-domain by one chosen out-of-domain assessment method does not imply that the point will be out-of-domain for all assessment methods. In summary, a user with limited trust on the model's predictions may raise similar concerns about the method used for out-of-domain assessment or provided explanatory methods. In a reductio ad absurdum, this process can go on indefinitely. Contrary to providing an explanation, one may consider the use of out-of-domain assessment a key value produced for a prediction. Such metrics are expected to provide enough information to guide the decision-making process, which is particularly relevant for sciences with high experimental costs or stringent regulatory acceptance criteria, such as the chemical, pharmaceutical and biological industries.

Neural networks have been frequently reported to be vulnerable to adversarial attacks, i.e., they may be exposed to sabotage attempts, such as deliberate reversal of a call or adding noise to the input representation. Beside the concern of safety, adversarial examples also play a role for applications with significantly lower levels of risk. Adversarial examples are usually defined as high-confidence misclassifications by the network. The most promising solutions use Monte Carlo Dropout networks. Even though, one may say there is one class for any image in the consideration, one should nonetheless keep in mind that the set of reported classes may be incomplete because an image can be perceived differently between humans. Consequently, we cannot always tell if the word negative originates from a true-negative call (“definitely not”) or a missed call (“forgot to mention”). In a first solution, Bayesian neural networks (BNN) have been used to evaluate the importance of uncertainty for adversarial examples. The evaluated BNNs display a significant shortcoming with respect to the concept of representation learning. Indeed, BNNs with their Bayesian logic are typically reported to be even less traceable than other neural networks. Consequently, the quality of the learnt representations cannot be judged and as such is an example of the beforementioned indefinability theorem. A meta-analysis is thus required to make a judgement call on the quality of the learnt representations. Albeit Monte Carlo dropout networks are significantly more robust against adversarial examples than their equivalent deterministic counterparts, these dropout networks are reported to typically underestimate uncertainty levels. In conclusion, even though it has been recognized that uncertainty and robustness are key points to combat adversarial examples, the quantification of the uncertainty levels is yet to be resolved.

In recent times and most notably in the context of deep fakes, adversarial neural networks are frequently used for generative purposes. The best-known example of such networks are generative adversarial networks (GAN). A typical GAN is composed of a system of two single deterministic networks, a generative neural network G and a discriminative neural network D. In analogy to the adversarial competition between the police force and criminal expert minds, GANs are trained applying an alternating mechanism of optimizing the generator G and the discriminator D. This alternating mechanism is applied with the aim that the generator G can produce realistic examples that are no longer distinguishable from real-world data by the discriminator D. GANs have been occasionally trained using an unsupervised discriminator. Irrespective of the chosen training mechanism, G and D define a single “machine” system. Consequently, quality and truth of the converged solution cannot be proven within the system itself. In other words, the fact of convergence of the model is not sufficient to proof that the produced output is indeed truly real. We thus need a meta-analysis to evaluate if the quality of such a neural network is within the expected limits and can be used as production application. An example of such meta-analysis is asking a representative set of users to select the real pictures from a mixed set of real and generated images.

Following on the above considerations, indefinability and incompleteness define key drawbacks for any machine learning model. In other words, the truth of one machine cannot be evaluated within the system itself. Even if a machine were to fail to answer certain questions, it does not mean that all machines would fail to answer the same set of wrongly answered questions. And, if for some problems, multiple machines provide correct answers, one cannot decide immediately what machine provides the better answers on all or parts of the questions asked. Lastly, one cannot know if the selected machine is the best solution for all future unseen data. We expect, however, that the selected machine is the best solution for most questions. One should nonetheless assume that the optimal results are usually obtained for the best performing model. To address this problem more deeply, it is essential to evaluate the predictive or model uncertainty. Additionally, one can consider that a trained model, which is typically composed of architecture, data, among some other variables, defines a closed system. The performance of this closed system is typically calibrated using a method of cross-validation. One should know that, even though the test data was not used for training, the test data is still a sample from currently known data, which has been produced for a specific purpose. Consequently, a bias may also be present in test data. We should thus anticipate that even if the test set is largely representative of a typical set of future unseen data points, this is not necessarily true for all future data. Consequently, results on future data may deviate significantly from the result measured on the test set. Strong deviations may be particularly in selection problems, where past selections are used to guide future selections. Additionally, the results are also strongly influenced by sample size and sample bias. Moreover, a validation of the model has already been performed during the training of the model using the test data and should not be judged using future data.

Uncertainties for modelling are typically split into aleatoric and epistemic uncertainties. Aleatoric uncertainty is in general understood as statistical uncertainty, i.e., it is affected by fluctuations produced by unknown effects every time an experiment has been run. More commonly, in this context one speaks about standard deviation or data variance. The aleatoric uncertainty is therefore defined by the data used to train the model and one refers to it as data uncertainty. One may thus expect that the prediction error is increasing with increasing noise levels in the data. One may also expect that data produced with the same procedure displays is homoscedastic, whereas data originating from multiple sources may be heteroscedastic. Changing fluctuations may also be introduced by experimental conditions that change over time. If the data is heteroscedastic, variance is an essential property to successfully merge data from different sources. Contrary to aleatoric uncertainty, one speaks about epistemic uncertainty to refer to the concept of errors induced by technical limitations or lack of knowledge in the case of machine-learning and deep-learning models. In modelling context, the epistemic uncertainty can thus be called model uncertainty. The epistemic error can be easily exemplified saying that this type of uncertainty is typically produced for cases where the model parameters are poorly determined, i.e., they have been trained with lack of data. Such case is also said to have a posterior that is broad on the hyperparameters of the network.

In recent years, different methods have been proposed to evaluate uncertainty in deep learning models. It should be noted that the assessment of uncertainty has been ongoing since the 1990s. These early publications do, however, refer to data uncertainty rather than model uncertainty. In other words, these models aim to also predict the variance observed in the data rather than the variance between models. A noticeable drawback of such a prediction is the fact that the data variance is typically not a property of the data point. In matter of fact, the observed data uncertainty may vary significantly based on the number of reported points and typically stabilizes to the true variance with increasing number of measurements. To assess the model uncertainty, different methods have been introduced. These methods are typically divided into four streams: 1) test-time augmentation methods, 2) Bayesian methods, 3) uncertainty for single deterministic neural networks, and 4) ensemble neural networks.

The uncertainty assessment produced with test-time augmentation methods is based on predicting different input representations using the same model. When speaking about augmentation, one speaks about generating different representations of the same object that can be sent into the same neural network. In case of images, augmentation can be easily exemplified by applying horizontal or verticals flips or rotations to the image. Irrespective of representation used, one expects the network to recognize the object. In this context, we prefer to speak about representation uncertainty. Test-time augmentation is thus an essential method for the evaluation of adversarial examples, i.e., we evaluate if modifications have no or limited impact on the model's predictions. In other reports, it has been demonstrated that augmentation should be applied during training-time and test-time to improve the robustness and stability of a neural network. The principal drawback for this method is the requirement that the data representation can be augmented. Whereas this is possible for image and language neural networks, augmentation is not possible for any network producing a latent space that is invariant to representation shuffle. An example of the latter type of neural networks are graph neural networks, which are frequently used for molecular predictions, such as shown in the paper Convolutional Networks on Graphs for Learning Molecular Fingerprints, by David Duvenaud & Al (https://arxiv.org/abs/1509.09292).

In Bayesian neural networks, a probability distribution over the model's parameters is learned, frequently followed by applying a normalization on these probabilities. The two consecutive steps create thus a layer of evidence on the essential features. A major drawback of the Bayesian approach primarily includes the fact that the solutions do not define a closed form solution, especially for systems with an enhanced level of complexity. Examples of complex systems are neural networks. To address this issue, computationally expensive approximative Bayesian inference (ABI) techniques need to be applied to compute the posterior probabilities. Alternately, ABI can be integrated into the network. This, however, systematically creates exceptionally large neural networks limiting the practical usability of such type of a network. Additionally, Bayesian neural networks also display a reduced level of traceability, which can be partially remedied by applying approximation techniques such as Monte Carlo approximations. Lastly, it is well known that the success of Bayesian approaches depends on the selection of relevant prior distributions. This point is yet to be resolved for neural networks. Specifying the optimal prior distributions remains an open challenge in deep learning context.

Application of Monte Carlo dropout strategies is currently the best available method to assess model uncertainty, such as shown in the paper Understanding Measures of Uncertainty for Adversarial Example Detection, by Lewis Smith & Al (https://arxiv.org/abs/1803.08533). In such a solution, the dropout layer is used during the training and inference stage of the model. To compute the uncertainty, multiple forward passes in the network are required defining a technical limitation to the method used. For the produced uncertainty values, it has been reported that these values are frequently too optimistic, more specifically it tends to underestimate the uncertainty. This effect can be explained with the fact that at every pass a subset of latent variables is randomly selected, and the random selection may repeatedly use the same set of variables. This problem can be remedied by using exceptionally large dropout rates, which as a matter of course leads to very large neural networks. It should be noted, that whereas the network size is less of a problem on images or texts, network size is a critical point to consider for applications with a considerable cost to produce additional data points.

Alternately, one can evaluate uncertainties in deep neural networks using single deterministic neural networks. In a first group of solutions, uncertainty can be estimated using, a second neural network, using a metric of distance to the training set or statistical hotelling of the latent space. None of the above methods takes influence on the already trained model. The indefinability problem is well visible in the concept of measuring distances to the training set. In this method, it is assumed that the distance is representative for the trust of a prediction. The produced distances, however, are a result from the selected definition and may vary significantly between definitions. In other words, the fact the distance is close in one definition, does not imply that the distance is close in all definitions. Moreover, a distance observed in one trained model may not even correspond to distances observed in a second model. In a reduction ad absurdum, one thus iteratively needs an additional system proving the results of the antecedent system. In summary, asking for an in-domain assessment, metric of trust or explainable Al is limited by the indefinability theorem.

### SUMMARY OF THE INVENTION

The present invention aims at addressing all or part of these drawbacks.

According to a first aspect, the present invention aims at a system for training an ensemble neural network device, comprising one or more computer processors and one or more computer-readable media operatively coupled to the one or more computer processors, wherein the one or more computer-readable media store instructions that, when executed by the one or more computer processors, cause the one or more computer processors to execute, the steps of:
providing a set of exemplar data, comprising at least one set of inputs and at least one set of outputs associated to the set of inputs, to a neural network device comprising an ensemble of neural network devices, configured to provide independent predictions based upon the exemplar data,
operating the neural network device based upon the set of exemplar data and
obtaining the trained neural network device configured to provide an output, in which:
the neural network device further comprises at least two independent activation functions, whereof at least two of the independent activation functions are representative of the statistical distribution of the plurality of independent predictions, the neural network device being configured to provide at least one output for at least two said independent activation functions and
the step of operating further comprising a step of operating each neural network device of the ensemble to provide an ensemble of outputs, the neural network device being trained to minimize the value representative of at least two said independent activation functions.

Such embodiments allow for much greater prediction stability, reliability, as well as improved training speed and overall performance and the provision of a metric of variance representative for the model uncertainty. Such embodiments thus allow resource savings, in terms of computation time or power, as well as in terms of model complexity. Typically, current approaches require the use of numerous models and iterations to obtain a reliable prediction model.

In such embodiments, for example the average of the distribution is trained to be minimized around a target and the variance of the distribution is trained to be minimized to a value close or equal to zero. This allows, in one call, to obtain the minimum variance possible on the trained ensemble neural network device.

In the prior art, the gradient of a loss function is sometimes modified as a function of the variance, which is based on the unverifiable hope that the loss function reduces the problem of scattered predictions, specifically this consists of solving simultaneity at least two equations in the loss. This creates a multi-objective optimization problem. Following the rules of logic, one has to find a compromised between those at least two equation objectives to be minimized and it's more complicated than having only one objective function to minimize. In particular embodiments, the neural network device obtained during the step of obtaining being configured to provide, additionally, a value representative of the dispersion of the output.

Such embodiments allow for greater model explainability.

In particular embodiments, at least two of the activation functions are representative of:
a means of the statistical distribution of the plurality of independent predictions and
the variance of the statistical distribution of the plurality of independent predictions.

In particular embodiments, the neural network device further comprises a layer configured to add simulacrums of outputs generated by using the learned distribution of the plurality of independent outputs as a function of the trained at least two of the at least two independent activation functions.

Such embodiments allow for the augmentation of the output so that the output is representative of the distribution in the event where the initial input is too small.

According to a second aspect, the present invention aims at a computer-implemented method to train a neural network device, comprising the steps of:
providing a set of exemplar data, comprising at least one set of inputs and at least one set of outputs associated to the set of inputs, to a neural network device comprising an ensemble of neural network devices, configured to provide independent predictions based upon the exemplar data,
operating the neural network device based upon the set of exemplar data and
obtaining the trained neural network device configured to provide an output,
the step of operating the neural network device, which further comprises at least two independent activation functions, whereof at least two of the independent activation functions are representative of a statistical distribution of the plurality of independent predictions, is configured to provide at least one output for at least two said independent activation functions and
the step of operating further comprising a step of operating each neural network device of the ensemble to provide an ensemble of outputs, the neural network device being trained to minimize the value representative of at least two said independent activation functions.

The method object of the present invention presents the same advantages as the system object of the present invention.

According to a third aspect, the present invention aims at a computer implemented neural network device obtained by the computer-implemented method object of the present invention.

The computer implemented neural network device object of the present invention presents the same advantages as the system object of the present invention.

According to a fourth aspect, the present invention aims at a computer program product, comprising instructions to execute the steps of a method object of the present invention when executed upon a computer.

The computer program product object of the present invention presents the same advantages as the system object of the present invention.

According to a fifth aspect, the present invention aims at a computer-readable medium, storing instructions to execute the steps of a method object of the present invention when executed upon a computer.

The computer-readable medium object of the present invention presents the same advantages as the system object of the present invention.

According to a sixth aspect, the present invention aims at a computer-implemented method to predict a physical, chemical, medicinal, sensorial, or pharmaceutical property of a flavor, fragrance or drug ingredient, comprising:
a step of training, by a computing device, a neural network device according to the method object of the present invention, in which the exemplar set of data is representative of:
   as input, compositions of flavor, fragrance, or drug ingredients and
   as output, at least one physical, chemical, medicinal, sensorial, or pharmaceutical property, one of said physical, chemical, medicinal, sensorial or pharmaceutical properties being the molecular weight of the composition,
a step of inputting, upon a computer interface, at least one flavor, fragrance or drug ingredient digital identifier, the resulting input corresponding to a composition of flavor, fragrance, or drug ingredients,
a step of operating, by a computing device, the trained neural network device trained and
a step of providing, upon a computer interface, for the composition, at least one physical, chemical, medicinal, sensorial, or pharmaceutical property output by the trained neural network device.

According to a seventh aspect, the present invention aims at a computer-implemented method to predict a category of representation in an image, comprising:
a step of training, by a computing device, a neural network device according to the method object of the present invention, in which the exemplar set of data is representative of:
   as input, images and
   as output, at least one category of representation in input images,
a step of inputting, upon a computer interface, at least one image,
a step of operating, by a computing device, the trained neural network device trained and
a step of providing, upon a computer interface, for the composition, at least one category of representation output by the trained neural network device.

Such provisions allow for predictions based on images, such as used in medical imagery or cellular activation for example.

### BRIEF DESCRIPTION OF THE DRAWINGS

Other advantages, purposes and particular characteristics of the invention shall be apparent from the following non-exhaustive description of at least one particular embodiment of the present invention, in relation to the drawings annexed hereto, in which:

FIG. 1 shows, schematically, a general representation of neural network device components,

FIG. 2 shows, schematically, a first particular succession of steps of the system object of the present invention,

FIG. 3 shows, schematically, a second particular succession of steps of the system object of the present invention,

FIG. 4 shows, schematically, a first particular succession of method of the system object of the present invention,

FIG. 5 shows, schematically, a first particular succession of method to predict a physical, chemical, medicinal, sensorial, or pharmaceutical property of a composition of fragrance or flavor ingredients object of the present invention,

FIG. 6 shows, schematically, a first particular succession of method to predict a category of representation of an image object of the present invention,

FIG. 7 shows, schematically, a computer architecture configured to execute a method object of the present invention,

FIG. 8 shows, schematically, two compared particular architectures used to predict the molecular weight of a molecule and

FIG. 9-13 show performance results of the two particular architectures.

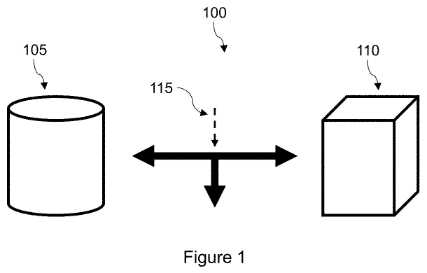

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted at this point that the figures are not to scale.

As used herein, the term “volatile ingredient” designates any ingredient, preferably presenting a flavoring or fragrance capacity. The terms “compound” or “ingredient” designate the same items as “volatile ingredient.” An ingredient may be formed of one or more chemical molecules.

The term composition designates a liquid, solid or gaseous assembly of at least one fragrance or flavor ingredient.

As used herein, a “flavor” refers to the olfactory perception resulting from the sum of odorant receptor(s) activation, enhancement, and inhibition (when present) by at least one volatile ingredient via orthonasal and retronasal olfaction as well as activation of the taste buds which contain taste receptor cells. Accordingly, by way of illustration and by no means intending to limit the scope of the present disclosure, a “flavor” results from the olfactory and taste bud perception arising from the sum of a first volatile ingredient that activates an odorant receptor or taste bud associated with a coconut tonality, a second volatile ingredient that activates an odorant receptor or taste bud associated with a celery tonality, and a third volatile ingredient that inhibits an odorant receptor or taste bud associated with a hay tonality.

As used herein, a “fragrance” refers to the olfactory perception resulting from the sum of odorant receptor(s) activation, enhancement, and inhibition (when present) by at least one volatile ingredient. Accordingly, by way of illustration and by no means intending to limit the scope of the present disclosure, a “fragrance” results from the olfactory perception arising from the sum of a first volatile ingredient that activates an odorant receptor associated with a coconut tonality, a second volatile ingredient that activates an odorant receptor associated with a celery tonality, and a third volatile ingredient that inhibits an odorant receptor associated with a hay tonality.

As used herein, the terms “means of inputting” is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

As used herein, the terms “computing system” or “computer system” designate any electronic calculation device, whether unitary or distributed, capable of receiving numerical inputs and providing numerical outputs by and to any sort of interface, digital and/or analog. Typically, a computing system designates either a computer executing a software having access to data storage or a client-server architecture wherein the data and/or calculation is performed at the server side while the client side acts as an interface.

As used herein, the terms “digital identifier” refers to any computerized identifier, such as one used in a computer database, representing a physical object, such as a flavoring ingredient. A digital identifier may refer to a label representative of the name, chemical structure, or internal reference of the flavoring ingredient.

As used herein, the terms “human reaction” refers to any physical behavior induced by confronting a human to a composition. This behavior may be broadly defined, such as appreciation or dislike for the composition or in more detail, such as describing facial expression or body movement when confronted with the composition.

In the present description, the term “materialized” is intended as existing outside of the digital environment of the present invention. “Materialized” may mean, for example, readily found in nature or synthesized in a laboratory or chemical plant. In any event, a materialized composition presents a tangible reality. The terms “to be compounded” or “compounding” refer to the act of materialization of a composition, whether via extraction and assembly of ingredients or via synthetization and assembly of ingredients.

As used herein, the terms “activation function” defines, in a neural network, how the weighted sum of the input is transformed into an output from a node or nodes in a layer of the network. These activation functions may be defined by layers in the network or by arithmetic solutions in the loss functions.

The embodiments disclosed below are presented in a general manner.

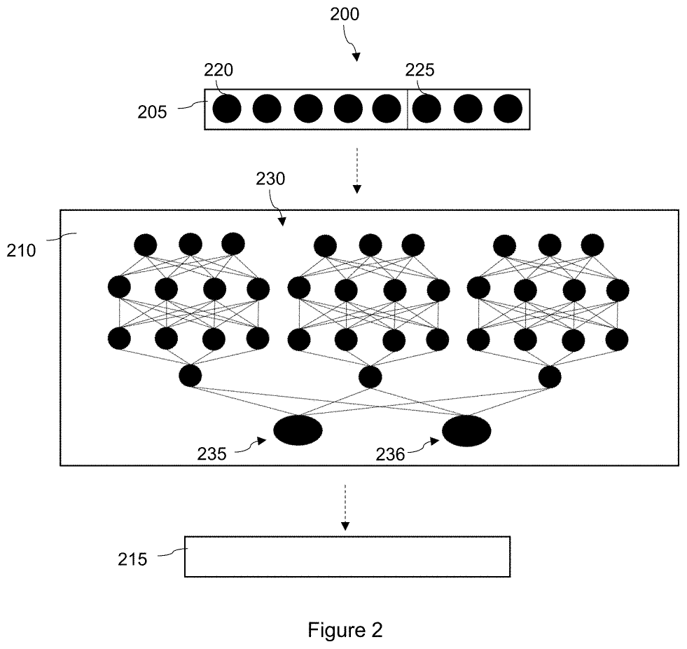

FIG. 2 shows a particular embodiment of the system 200 object of the present invention. This system 200 for training an ensemble neural network device, comprising one or more computer processors and one or more computer-readable media operatively coupled to the one or more computer processors, wherein the one or more computer-readable media store instructions that, when executed by the one or more computer processors, cause the one or more computer processors to execute the steps of:
providing 205 a set of exemplar data, comprising at least one set of inputs 220 and at least one set of outputs 225 associated to the set of inputs, to a neural network device comprising an ensemble 230 of neural network devices, configured to provide independent predictions based upon the exemplar data,
operating 210 the neural network device based upon the set of exemplar data and
obtaining 215 the trained neural network device configured to provide an output, in which:
the neural network device further comprises at least two independent activation functions, whereof at least two of the independent activation functions are representative of the statistical distribution of the plurality of independent predictions, the neural network device being configured to provide at least one output, 235 and 236, for at least two said independent activation functions and
the step of operating further comprising a step of operating each neural network device of the ensemble to provide an ensemble of outputs, the neural network device being trained to minimize the value representative of at least two said independent activation functions.

The system 200 as such may be formed of any combination of means to execute the characteristic steps executed by the computer processors.

The step 205 of providing may be performed, via a computer interface, such as an API or any other digital input means. This step 105 of providing may be initiated manually or automatically. The set of exemplar data may be assembled manually, upon a computer interface, or automatically, by a computing system, from a larger set of exemplar data.

The exemplar data may comprise, for example:
at least one at least one fragrant, flavor or drug ingredient digital identifier, said at least one at least one fragrant, flavor or drug ingredient digital identifier forming a composition, said composition being optionally associated with a composition identifier and
a molecular weight for the composition.

Such a set of exemplar data may be obtained by assembling compositions and mathematically adding the theoretical weight of each atom to obtain the molecular weight of compositions of the exemplar set.

In other variants, the exemplar data may comprise, for example:
at least one image and
for at least one said image, a category among a list of possible categories of what the image represents (‘airplane’, ‘car’, bird′, ‘cats’, ‘deer’, ‘dog’, ‘frog’, ‘horse’, ‘ship’, and ‘truck’, for example). Such a category is sometimes called a tag, a label, a call, or a class of representation.

The step 110 of operating may be performed, for example, by a computer program executed upon a computing system. During this step 110 of operating, the ensemble neural network device is configured to train based upon the input data. During this step 110 of operating, each neural network of the ensemble neural network device configures coefficients of the layers of artificial neurons to provide an output, these outputs forming a distribution of outputs. Values of statistical parameters representative of the distribution may be obtained and used in activation functions to be minimized.

In particular embodiments, at least two of the activation functions are representative of:
a means of the statistical distribution of the plurality of independent predictions,
the variance of the statistical distribution of the plurality of independent predictions and
optionally extended with additional activation functions, representative of:
   the skew of the statistical distribution of the plurality of independent predictions and/or
   the kurtosis of the statistical distribution of the plurality of independent predictions.

The step 115 of obtaining may be performed, via a computer interface, such as an API or any other digital output system. The obtained trained ensemble neural network device may be stored in a data storage, such as a hard-drive or database for example.

In particular embodiments, the neural network device obtained during the step 215 of obtaining being configured to provide, additionally, at least one value representative of the statistical dispersion of the output.

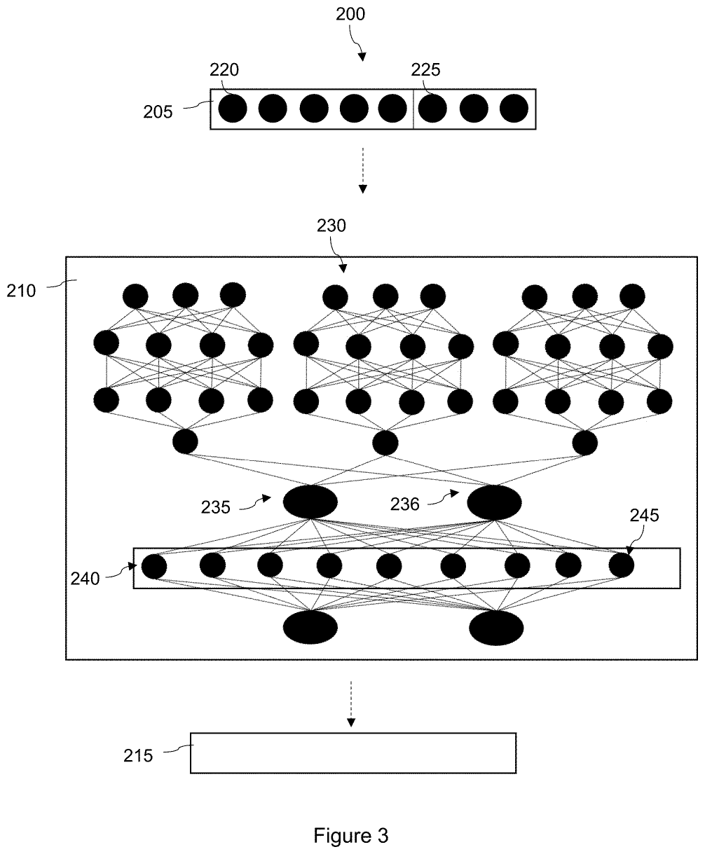

FIG. 3 shows a particular embodiment of the system 200 object of the present invention.

In this embodiment, the neural network device further comprises a layer 240 configured to add simulacrums 245 of outputs generated by using the learned distribution of the plurality of independent outputs as a function of the trained at least two of the at least two independent activation functions.

Such embodiments may correspond, for example, to a Gaussian augmentation of the output, based upon the means and variance of the output that the neural network device provides.

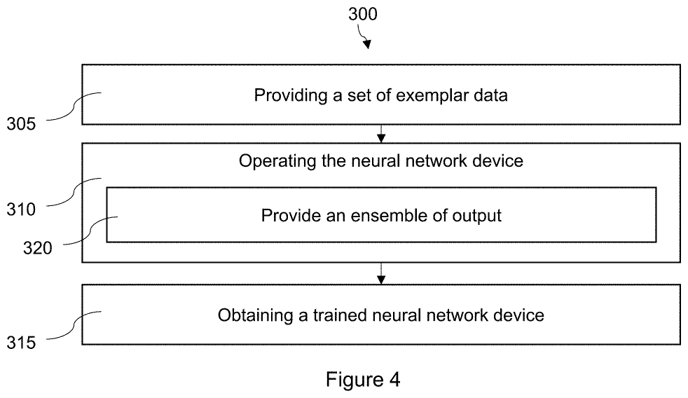

FIG. 4 represents, schematically, a particular succession of steps of the computer-implemented method 300 to train a neural network device, comprising the steps of:
providing 305 a set of exemplar data, comprising at least one set of inputs and at least one set of outputs associated to the set of inputs, to a neural network device comprising an ensemble of neural network devices, configured to provide independent predictions based upon the exemplar data,
operating 310 the neural network device based upon the set of exemplar data and
obtaining 315 the trained neural network device configured to provide an output, in which:
the step of operating the neural network device, which further comprises at least two independent activation functions, whereof at least two of the independent activation functions are representative of a statistical distribution of the plurality of independent predictions, is configured to provide at least one output for at least two said independent activation functions and
the step of operating further comprising a step 320 of operating each neural network device of the ensemble to provide an ensemble of outputs, the neural network device being trained to minimize the value representative of at least two said independent activation functions.

The steps of providing 305, operating, 310 and 320, obtaining 315 are disclosed in regard to the corresponding steps of the system 200 object of the present invention shown in FIGS. 2 and 3.

The present invention aims at a computer implemented neural network device, characterized in that the neural network device is obtained by the computer-implemented method 300 according to claim 5.

The present invention aims at a computer program product, comprising instructions to execute the steps of a method 300 such as shown in FIG. 4 when executed upon a computer.

The present invention aims at a computer-readable medium, storing instructions to execute the steps of a method 300 such as shown in FIG. 4 when executed upon a computer.

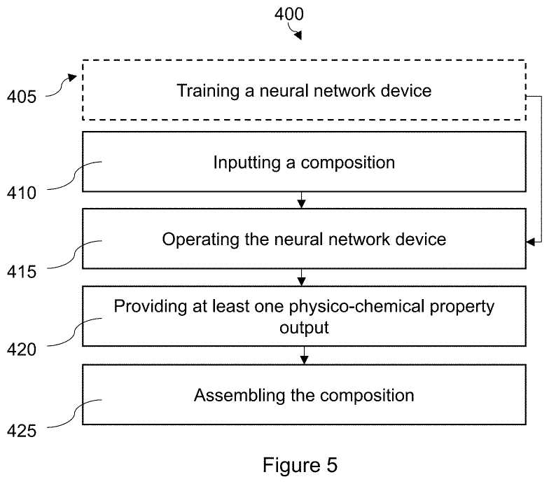

FIG. 5 represents, schematically, a particular succession of steps of the method 400 object of the present invention. This computer-implemented method 400 to predict a physical, chemical, medicinal, sensorial, or pharmaceutical property (such as disclosed in the publication Molecular descriptors for chemoinfomatics Roberto Todeschini, Viviana Conesonni second, revised and enlarged edition, by Wiley et al.) of a composition of flavor, fragrance, or drug ingredients, comprises:
a step 405 of training, by a computing device, a neural network device according to the method 300 such as shown in FIG. 4, in which the exemplar set of data is representative of:
   as input, compositions of flavor, fragrance, or drug ingredients and
   as output, at least one physical, chemical, medicinal, sensorial or pharmaceutical property, one of said physical, chemical, medicinal, sensorial or pharmaceutical properties being the molecular weight of the composition,
a step 410 of inputting, upon a computer interface, at least one flavor, fragrance or drug ingredient digital identifier, the resulting input corresponding to a composition of flavor, fragrance, or drug ingredients,
a step 415 of operating, by a computing device, the trained neural network device trained and
a step 420 of providing, upon a computer interface, for the composition, at least one physical, chemical, medicinal, sensorial, or pharmaceutical property output by the trained neural network device.

The method 400 object of the present invention as one of the embodiments of the system 200 object of the present invention disclosed in regards of FIG. 2.

It is thus possible predict the molecular weight (MW) of a molecule. A molecular weight is computed by summing the atomic weight over all atoms in the molecule. By using such simple target one can evaluate if a given architecture can extract the meaningful molecular information from any data size considering that we can compute the molecular weight for any given proposed molecule. A major advantage of this approach includes the fact that one has an exact metric showing no variance on the measurement. Contrary to molecular weight, an experimentally measured target is by essence not just a sum of atomic weights but rather a complex function on the conditions used. If we can accurately predict molecular weight, we can at least validate that a model has correctly extracted the chemical knowledge from the data, considering the uncertainty drawbacks listed above, an evaluation of the chemical knowledge extraction is not trivial if a model is exclusively trained on an experimental target.

Below, further considerations and embodiments are disclosed:

Relative to chemical properties, are presented herein the results on the prediction of molecular weight in a neural network of the present invention. Albeit the task of predicting molecular weight seems obvious, the prediction of the molecular weight has two principal advantages: Firstly, the target is an exact value with minimal to no variance on the value. In the experiments one can thus assess the results excluding the data variance as explanation for the results. Secondly, the prediction clearly communicates whether a neural network has been able to make the correct chemical abstraction.

This comparison is performed for the prediction of molecular weight by a single deterministic neural network (SDNN) and an ensemble neural network trained using mean and variance (MSENN). The models have been trained using a recurrent neural network, typically used in natural-language processing. The input is defined by tokenized vector defining the atoms and bonds in the molecule, closely resembling the tokenized input typically used for natural language processing. An example of such format are SMILES strings. The results have been computed for an internal dataset for 9979 molecules found typically in natural plants used for their olfactive, taste and medicinal properties with molecular weight <450.

As a first result, the results shows that the reproducibility of SDNNs is limited. Firstly, one can see that the performance of SDNNs fluctuate strongly. Indeed, even though the variance on the data is limited, large differences can be seen for the RMSE on both the train set 905 and test set performances 910. Please note, that all networks have been trained starting from the identical initial weights. Secondly, one observes that the performance is strongly dependant on the used data split. As a matter of fact, one can observe that for some data splits the test performance is too optimistic, for some points well equilibrated, and, too pessimistic for most splits. Based on the fluctuating results, one can already define that the expected performance may vary significantly. In other words, the performance obtained on one test set is not indicative to know the performance on another test set. Even though one expects that the performance on future unseen data may display the same performance variations, the exact performance is strongly dependent on the evaluated selection. Indeed, sample size and sample bias on future selected datasets will strongly influence the performance.

As a second result, the performance of our ensemble neural networks is compared against the performance of SDNNs applying the same train-test splits between the two networks. Firstly, one can see that with one exception, the performance 1005 on the training sets is stable with RMSE between 0.5 to 0.6. The distribution with observed RMSE values on the train and test sets shows clear downshifts 1010 for the ensemble models in comparison with single models. Secondly, one can see that also the performance for the test set has significantly improved compared to the performance on a single network. As mentioned previously, the performance varies depending on split used. Indeed, even though the training performance is very robust in the present ensemble neural networks, the range of test performances 1015 is very large. These results once again suggest that the performance on one test is not indicative for the performance on another test. As mentioned previously, one should also expect similar variations when validating the performance on future unseen data. Thirdly, one can also see that the neural networks have decreased the RMSE by 15-30% when using ensemble neural networks. The reduction is observed for both the train and test sets in the data, 1020 and 1025.

In summary, an ensemble neural network which is actively trained on mean and variance shows better performances compared to single deterministic neural networks.

While a normal distribution training the mean and variance can be used, this method can be used with other statistical distributions.

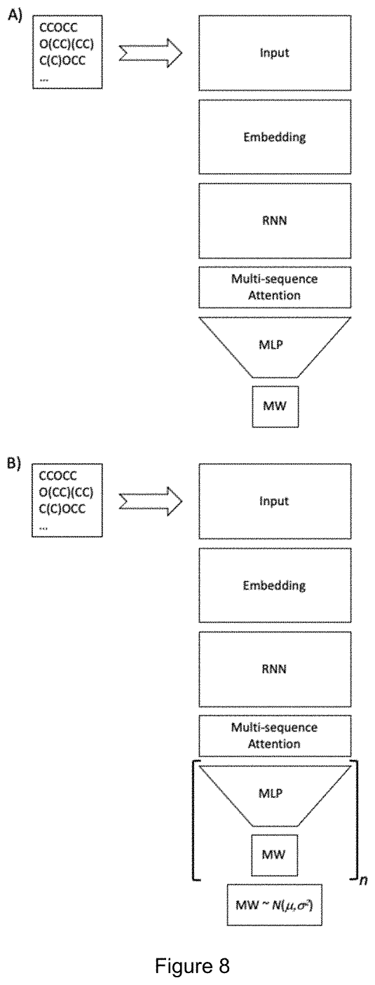

FIG. 8 shows two neural network devices trained to predict the molecular weight of a molecule. For both networks the input is a set of multiple NLP-compatible representation defining the same molecule as shown for the molecule diethyl ether. The Embedding layer is a conversion from discrete integers to continuous vectors. The RNN layer applies a temporal sequence analysis. Examples of layers are GRU or LSTM. Multi-sequence attention defines a pooling mechanism to combine the acquired knowledge from all processed input sequences. MLP defines a multi-layer perceptron composed of multiple fully connected layers of reducing size activated with ‘selu’ activation function other activation function like ‘luckyrelu’, ‘relu’, ‘elu’ for example could be used. MW defines the layer prediction the target value. A) Architecture for a single deterministic neural network. This network is trained on the expected value. B) Architecture for our ensemble composed of an ensemble of n MLPs producing MW as a normal distribution N(μ, σ²). The model is trained to minimize the mean and variance to the expected value and 0, respectively.

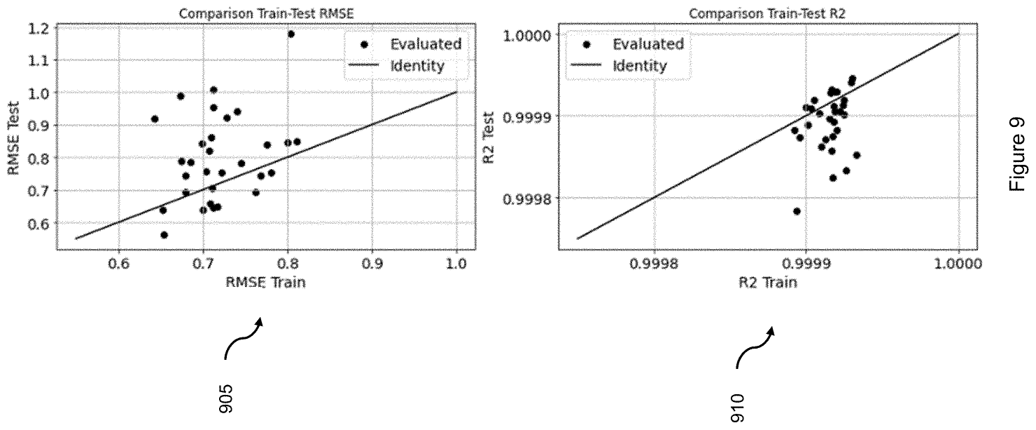

FIG. 9 shows the performance of molecular weight prediction by a single deterministic network. The values shown are root-mean-squared-error (RMSE) and coefficient of correlation (R2). The black line shows the case of equal RMSE or R2 between train and test. A) Results shown for RMSE(test) vs RMSE(train). B) Results shown for R2(test) vs R2(train).

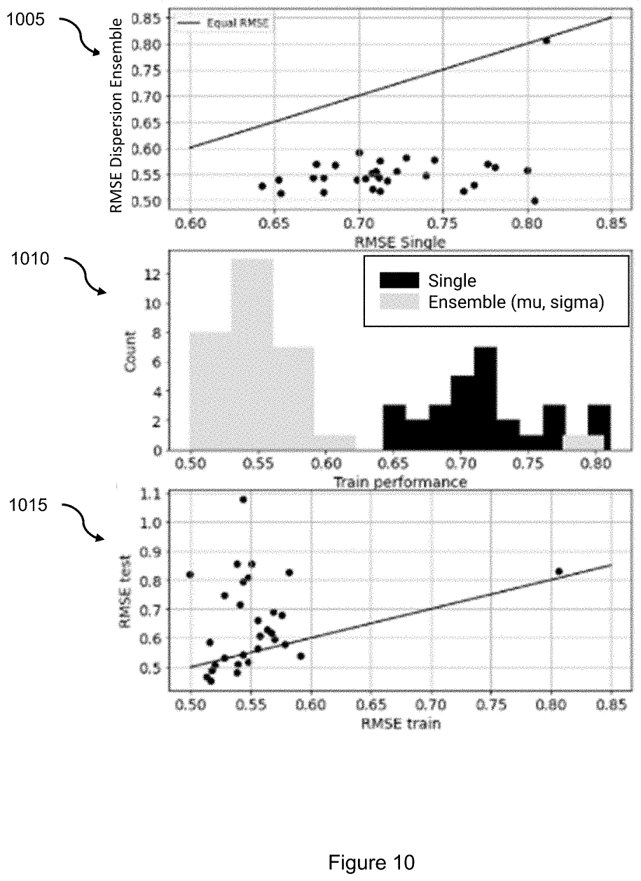

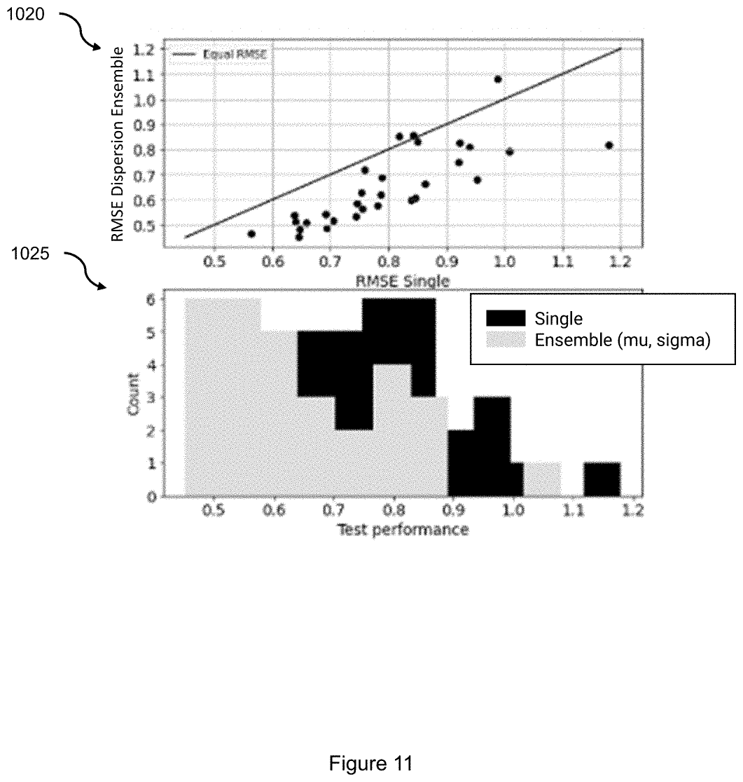

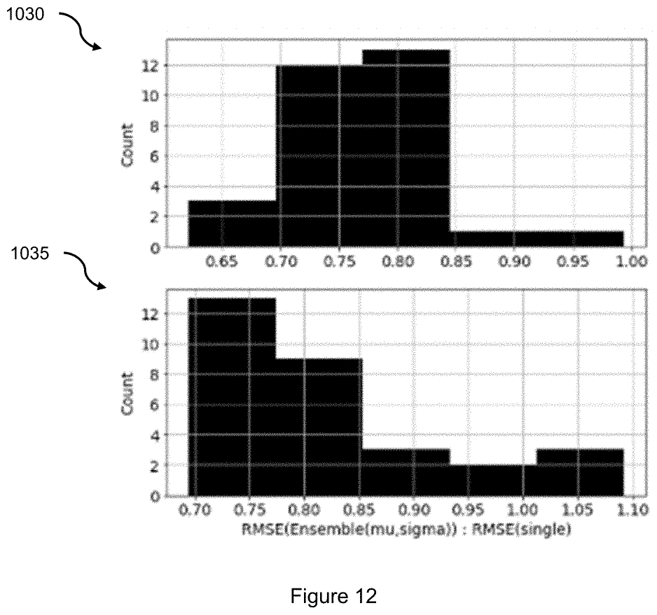

FIGS. 10 to 12 show the performance of molecular prediction by the ensemble neural network device of the present invention, indicated by “Dispersion Ensemble”, while a single deterministic neural network is indicated with Single. Reference 1005 shows the comparison of RMSE(Dispersion Ensemble) vs RMSE(Single) on the training set for each split. Reference 1020 shows the comparison of RMSE(Dispersion Ensemble) vs RMSE(Single) on the test set for each split. Reference 1030 shows a histogram of the RMSE for the training sets, showing Single (black) and Dispersion Ensemble (grey). Reference 1010 shows a histogram of the RMSE for the test sets, showing Single (black) and Dispersion Ensemble (grey). Reference 1025 shows an error reduction analysis on the performance of the training sets calculates as RMSE(Dispersion Ensemble):RMSE(Single), i.e., values <1.0 show an improvement. Reference 135 shows an error reduction analysis on the performance of the test sets calculates as RMSE(Dispersion Ensemble):RMSE(Single), i.e., values <1.0 show an improvement. Reference 1015 shows the performance of RMSE(test) vs RMSE(train) for our ensemble neural network Dispersion Ensemble.

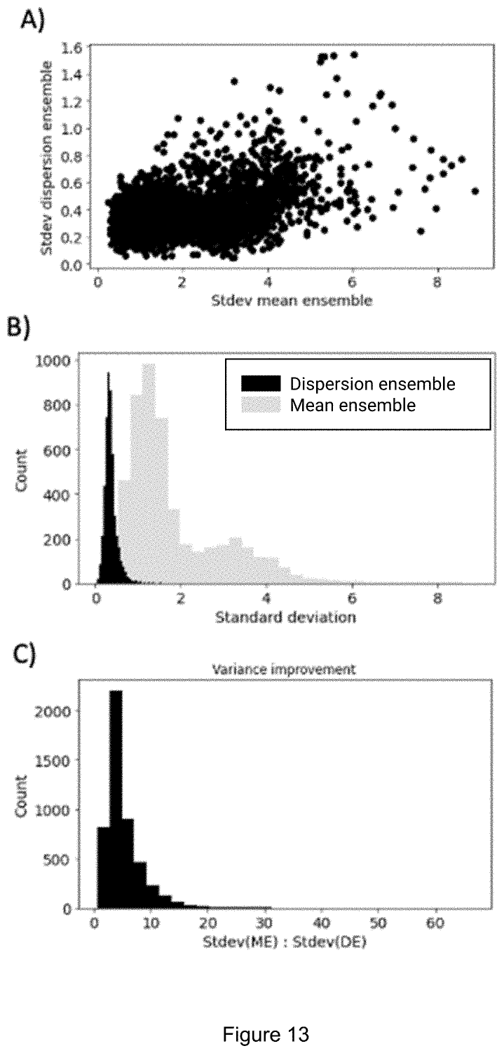

FIG. 13 shows the comparison of mean-ensemble and the preset dispersion ensemble. One can compare the predictions of a classical mean-ensemble with the present dispersion ensemble using the same splits, considering all molecules with a molecular weight (MW)<450. The herein proposed dispersion ensemble predicts the MW with a remarkable improvement on the precision, i.e., uncertainty, of the prediction. In the mean ensemble, the model reports a standard deviation of 4 (95% cutoff), corresponding to a difference of 8 hydrogen atoms. The dispersion ensemble model, however, shows a standard deviation of 0.8 (95%) corresponding to a difference of <2 hydrogens. Knowing the relevant number of hydrogens defines an important information for the level of saturation in the molecule.

FIG. 13 indeed shows the prediction comparison between mean-ensemble and dispersion ensemble for the prediction of the molecular weight (MW). A) Scatter plot of standard deviation (dispersion ensemble, y-axis) vs standard deviation (mean-ensemble, x-axis). B) Distributions of the reported standard deviation values on inference for the dispersion ensemble (black) and mean-ensemble (grey). C) Histogram showing the improvement on the reported standard deviation. The standard deviation of the mean-ensemble is divided by standard deviation of the dispersion ensemble.

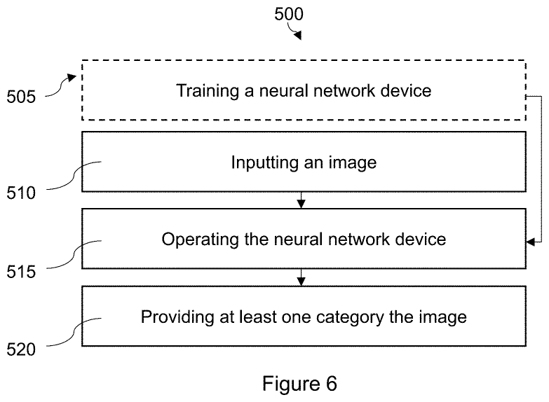

FIG. 6 represents, schematically, a particular succession of steps of the method 500 object of the present invention. This computer-implemented method 500 to predict a category of representation in an image comprises:
a step 505 of training, by a computing device, a neural network device according to the method 300 such as shown in FIG. 4, in which the exemplar set of data is representative of:
   as input, images and
   as output, at least one category of representation in input images,
a step 510 of inputting, upon a computer interface, at least one image,
a step 515 of operating, by a computing device, the trained neural network device trained and
a step 520 of providing, upon a computer interface, for the composition, at least one category of representation output by the trained neural network device.

The method 500 object of the present invention as one of the embodiments of the system 200 object of the present invention disclosed in regards of FIG. 2.

Below, further considerations and embodiments are disclosed:

Neural networks are an emerging trend being introduced for a wide range of applications. Based on a large volume of images, neural networks have been widely adopted for image problems. Frequently, neural network image learning can be easily resolved using single deterministic neural networks. The principal drawback of single deterministic neural networks is that these neural networks do not communicate the predictive model uncertainty. A second drawback of these networks is that most single networks are not robust, making them highly vulnerable to data perturbations, more colloquially known as adversarial examples. In summary, assessment of model uncertainty has been recognized as one of the key areas that is not yet resolved.

Recently, the use of evidential deep learning has been introduced to estimate the model uncertainty for image classification. In this method, a single additional evidential layer is introduced to provide the parameters for a pre-selected distribution. The variance can then be mathematically computed applying the variance equation for the selected distribution. In the work by Sensoy et al. (https://arxiv.org/abs/1806.01768), a Dirichlet distribution has been used as supplier of the variance. This has led to the detection of out-of-domain queries and increased robustness against adversarial perturbations. A first major drawback of the method is introduced with the selection of the distribution. The resulting variance is now thus a bound result of the trained system. In a reductio ad absurdum, one may even say that the solution of the variance, is equally subject to the same concerns. Indeed, the new parameters are also originating from a new single deterministic neural network.

To remedy these drawbacks, the present invention uses an ensemble neural network (ENN). ENNs have been introduced to improve the robustness of neural network, but also to provide a notion of model uncertainty. Examples of ENNs are test-time mean ensembles, bootstrapping ensembles, snapshot ensembles, dropout ensembles, mean ensembles, mean-variance ensembles and ensembles trained using negative correlation learning.

In this group, snapshot ensembles and dropout ensembles stand out, because they are typically computed using a single deterministic neural network. In a snapshot ensemble, multiple weight configurations at varying time points are combined. The resulting variance is thus a metric of time stability for the predicted point. In a dropout ensemble, an uncertainty is produced by applying the dropout layer also on inference time. The produced variance is thus a measure of stability for parameter subsampling. A drawback of both networks is that the predicted uncertainty is frequently underestimated. In snapshot ensembles this is a result from a time dependence. In dropout ensembles, variables may be present in multiple selections. This can be remedied by applying neural networks with very high dropout rates. This, however, may have significant influence on the size of the network.

A particular solution is provided by bootstrapping ensembles. In this type of network an ensemble is created by training the same network with different data selections. The notion of the produced model uncertainty is thus a metric or robustness against data subsampling. In this type of ensemble, high density points are well supported and are not affected by subsampling. The same concern raised for dropout networks, can be raided for bootstrapping ensembles. It is expected that the uncertainty might be underestimated because small bootstrapping omission rates may yield to repeated use of data points. The latter is particularly beneficial for points originating from high density rates in the data.

The group of mean ensembles and test-time mean ensembles define a group of ensemble networks that are combined to produce a mean and variance for the predictions. Whereas the mean ensemble is actively trained on the mean, a test-time ensemble is an ensemble of independently trained networks. In the case of mean networks, the model is trained on predicted the right mean value. A major drawback of only training the mean is the fact that the submodels' variance may vary significantly between predictions on different points. In a test-time ensemble, all models are individually trained. Consequently, these networks may even display issues that the mean value between the networks is not even optimized as it has been done for the mean ensemble counterparts.

Mean-variance ensemble and lower upper bound ensemble neural networks are trained using the data variance. The lower and upper bounds are a variance of the mean and variance, i.e., the lower and upper bounds have been computed as mean-variance and mean+variance, respectively. In these networks, the network is trained using the data variance. The major drawback in this approach is that the variance does not provide any conclusion on the model uncertainty. Additionally, the data variance itself is not a property of the predicted point, but a property of observed fluctuations on its measurements. In matter of fact, the reported variance strongly depends on the number of measurements performed for each data points. The number of reported data points may vary significantly from point to point.

The drawbacks mentioned previously have been resolved applying a strategy called negative correlation learning. In this approach, one typically modifies the loss to account for the diversity in the signal. Examples of proposed training mechanisms are the use of a coupling term or the use of KL-divergence. The methods have been extensively evaluated and it has been observed that the performance of the base learners is strongly varying. Whereas the method is usually beneficial for small-capacity base learners, the method is reported to be harmful for large-capacity base learners. In summary, the use of NCL in ensembles requires hard fine-tuning optimization to hopefully reach good results.

When training a single deterministic neural network, one commonly cannot tell if the initialization of such a network may lead to the best result. In addition, one cannot exactly if the model has developed a bias for a particular subset of the used data. In extreme cases, one may observe that the model may fail to provide answers to some questions asked, i.e., it may fail to correctly predict some points.

In this particular embodiment, an ensemble neural network device is trained on both the mean and variance to establish a communication between the submodels of an ensemble neural network. As a simplification for the training mechanism, a sampling mechanism is applied on the mean and variance produced in the ensemble, much like the sampling mechanism used in variational auto-encoders (VAE).

Note that, contrary to the present system, a VAE is a single deterministic neural network using an independent layer of random variance to become a generative neural network by applying the sampling mechanism.

In this work, the methodology has been applied to image classification using the CIFAR-10 dataset. In CIFAR-10, one asks the models to predict one class from 10 possible classes for a set of images. The results are computed for 5 different splits with a training size of 50,000 images and a test set of 10,000 images. The results have been summarized by measuring the classification accuracy, i.e., the percentage of correct predictions.

The comparison of performances on a network sampling using the ensemble's mean and variance, sampling using a full covariance in the ensemble, and sampling from an independent layer of variance produced in the network is obtained. Note that the latter method is identical to the strategy used in VAEs. In the table below, the three methods are referred to as Diagonal, Full Covariance, and Diagonal MLP, respectively. The present methods have been compared to five existing solutions: 1) mean ensemble, 2) negative-correlation learning, 3) single deterministic neural network, 4) dropout ensemble, and 5) bootstrapping ensembles. In this table, these solutions are identified by Mean ensemble, NCL, Single deterministic NN, Dropout ensemble and Bagging ensemble respectively. The reported results are prediction accuracy.

Performance results on the tested methodologies, sorted by decreasing accuracy.

| Methodology | Validation accuracy |
|---|---|
| Full covariance (present invention) | 83.1 +/− 0.3% |
| Dropout ensemble | 82.8 +/− 1.1% |
| Diagonal (present invention) | 82.4 +/− 0.2% |
| Negative correlation learning NCL | 81.7 +/− 0.4% |
| Bagging ensemble | 81.6 +/− 0.2% |
| Mean ensemble | 79.2 +/− 0.3% |
| Single deterministic NN | 77.0 +/− 0.5% |
| Diagonal MLP (present invention) | 76.0 +/− 0.5% |

The results in the above table show some clear results. Firstly, all ensemble neural networks outperform the single deterministic neural networks. Indeed, the networks Single and Diagonal MLP display significantly lower performances than the six tested ensemble methodologies on top of the table. Secondly, for Diagonal MLP one can see that the use of an independent layer of random variance is not beneficial to improve results. Moreover, the results show that the performance drop in Diagonal MLP is statistically significant when compared to Single. Thirdly, one can see that the classical mean ensemble Mean, the bootstrapping ensemble Bagging and the negative correlation learning NCL can all improve the prediction accuracies. Fourthly, one can observe that the present ensemble techniques Full Covariance and Diagonal perform significantly better. Fifthly, from the reported ensemble methods, only the dropout ensembles can reach similar accuracy performances. It should be noted, however, that the dropout ensembles show however strong fluctuations on the reported performances. Whereas our ensemble methodologies Full covariance and Diagonal show variance of 0.3% and 0.2%, respectively, the dropout ensembles show a significantly larger variance of 1.1% showing a stronger robustness than the dropout existing method.

In summary, ensemble neural networks with communicating submodels reach consensus agreements that outperform previously reported ensemble neural networks on prediction accuracy reproducibility.

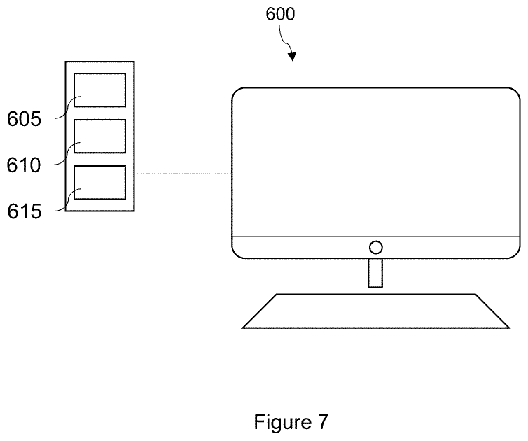

FIG. 7 represents, schematically, a computer architecture 600 capable of implementing the system 200 object of the present invention. This computer architecture 600 comprises:
means to provide a set of exemplar data, comprising at least one set of inputs and at least one set of outputs associated to the set of inputs, to a neural network device comprising an ensemble of neural network devices, configured to provide independent predictions based upon the exemplar data,
means to operate the neural network device based upon the set of exemplar data and
means to obtain the trained neural network device configured to provide an output, in which:
the neural network device further comprises at least two independent activation functions, at least two of the at least two independent activation functions being representative of the dispersion in a statistical distribution of the plurality of independent predictions, the neural network device being configured to provide at least one output for at least two said independent activation functions and
means of operating further comprising means of operating each neural network device of the ensemble to provide an ensemble of outputs, the neural network device being trained to minimize the value representative of at least two said independent activation functions.

1. System for training an ensemble neural network device, comprising one or more computer processors and one or more computer-readable media operatively coupled to the one or more computer processors, wherein the one or more computer-readable media store instructions that, when executed by the one or more computer processors, cause the one or more computer processors to execute steps of:
providing a set of exemplar data, comprising at least one set of inputs and at least one set of outputs associated to the set of inputs, to a neural network device comprising an ensemble of neural network devices, configured to provide independent predictions based upon the exemplar data,
operating the neural network device based upon the set of exemplar data and
obtaining the trained neural network device configured to provide an output, wherein:
the neural network device further comprises at least two independent activation functions, whereof at least two of the independent activation functions are representative of the statistical distribution of the plurality of independent predictions, the neural network device being configured to provide at least one output for at least two said independent activation functions and
the step of operating further comprising a step of operating each neural network device of the ensemble to provide an ensemble of outputs, the neural network device being trained to minimize the value representative of at least two said independent activation functions.

2. System according to claim 1, in which the neural network device obtained during the step of obtaining being configured to provide, additionally, a value representative of the dispersion of the output.

3. System according to claim 1, in which at least two of the activation functions are representative of:
a means of the statistical distribution of the plurality of independent predictions and
the variance of the statistical distribution of the plurality of independent predictions.

4. System according to claim 1, in which the neural network device further comprises a layer configured to add simulacrums of outputs generated by using the learned distribution of the plurality of independent outputs as a function of the trained at least two of the at least two independent activation functions.

5. Computer-implemented method to train a neural network device, comprising the steps of:
providing a set of exemplar data, comprising at least one set of inputs and at least one set of outputs associated to the set of inputs, to a neural network device comprising an ensemble of neural network devices, configured to provide independent predictions based upon the exemplar data,
operating the neural network device based upon the set of exemplar data and
obtaining the trained neural network device configured to provide an output, wherein:
the step of operating the neural network device, which further comprises at least two independent activation functions, whereof at least two of the independent activation functions are representative of a statistical distribution of the plurality of independent predictions, is configured to provide at least one output for at least two said independent activation functions and
the step of operating further comprising a step of operating each neural network device of the ensemble to provide an ensemble of outputs, the neural network device being trained to minimize the value representative of at least two said independent activation functions.

6. Computer implemented neural network device, wherein the neural network device is obtained by the computer-implemented method according to claim 5.

7. Computer program product, which comprises instructions to execute the steps of a method according to claim 5 when executed upon a computer.

8. Computer-readable medium, which stores instructions to execute the steps of a method according to claim 5 when executed upon a computer.

9. Computer-implemented method to predict a physical, chemical, medicinal, sensorial, or pharmaceutical property of a flavor, fragrance or drug ingredient, which comprises:
a step of training, by a computing device, a neural network device according to the method object of claim 5, in which the exemplar set of data is representative of:
as input, compositions of flavor, fragrance, or drug ingredients and
as output, at least one physical, chemical, medicinal, sensorial, or pharmaceutical property, one of said physical, chemical, medicinal, sensorial, or pharmaceutical properties being the molecular weight of the composition,
a step of inputting, upon a computer interface, at least one flavor, fragrance or drug ingredient digital identifier, the resulting input corresponding to a composition of flavor, fragrance, or drug ingredients,
a step of operating, by a computing device, the trained neural network device trained and
a step of providing, upon a computer interface, for the composition, at least one physical, chemical, medicinal, sensorial, or pharmaceutical property output by the trained neural network device.

10. Computer-implemented method to predict a category of representation in an image, which comprises:
a step of training, by a computing device, a neural network device according to the method object of claim 5, in which the exemplar set of data is representative of:
as input, images and
as output, at least one category of representation in input images,
a step of inputting, upon a computer interface, at least one image,
a step of operating, by a computing device, the trained neural network device trained and
a step of providing, upon a computer interface, for the composition, at least one category of representation output by the trained neural network device.
